(19) United States
(12) Patent Application Publication
(10) Pub. No.: US 2002/0068011 A1
(43) Pub. Date: Jun. 6, 2002

(54) Method and apparatus for sterilizing infectious wastes on site

(75) Inventor: Nils E. Kongmark, Sauverny (FR)

Correspondence Address: D. Peter Hochberg Co., L.P.A., 6th Floor, 1940 East 6th Street, Cleveland, OH, 44114-2294, US

(73) Assignee: R.I.M.M. Technologies N.V.

(21) Appl. No.: 09/943,532

(22) Filed: Aug. 30, 2001

Related U.S. Application Data
(63) Continuation-in-part of application No. 09/180,971, filed on Jan. 13, 1999, now abandoned.
Application No. 09/180,971 is a 371 of international application No. PCT/IB97/00532, filed on May 9, 1997.

(30) Foreign Application Priority Data
May 17, 1996 (CH) 1248/96

(51) Int. Cl. A61L 2/00; A61L 9/00

(52) U.S. Cl. 422/21; 422/32

(57) ABSTRACT

The invention discloses a method for on-site treatment of medical and veterinary wastes by sterilization, comprising the following successive steps: preparing the wastes for subsequent handling and treatment by electromagnetic heating; dividing the wastes into homogeneous batches of substantially equal sizes; sterilizing the waste batches by an electromagnetically generated heat source; aseptically transporting the sterilized wastes outside the treatment zone and retrieving them for transportation without outside exposure. The preparation of the wastes comprises a step of crushing, moistening or wetting. This method, enabling the on-site treatment of medical and veterinary wastes, avoids contamination risks connected with untreated waste transportation. The invention also features an apparatus for implementing the method.

### CROSS-REFERENCE TO RELATED APPLICATION

This application is a continuation-in-part of U.S. Ser. No. 09/180,971 filed Jan. 13, 1999 which is the U.S. national phase of PCT/IB97/00532 filed on May 9, 1997.

### BACKGROUND OF THE INVENTION

1. Field of the Invention

The present invention relates to a method and apparatus for sterilizing wastes on site, particularly infectious wastes such as medical and veterinary waste, in view of their environmentally safe disposal.

2. Description of the Prior Art

The treatment and elimination of medical and veterinary wastes is an acutely increasing problem, especially for hospitals and clinics. All the known methods of treatment are based on the removal of the wastes from the place where they were created to stock them in a place where they are considered harmless. The problem is that these wastes can cause contamination during transport, not only because the confinement means during transport can fail, but also because of a simple human factor. The best method would consist in sterilizing infectious wastes, which represents approximately 20% of the total medical and veterinary wastes, on site where they are produced. However, no satisfactory solution of this type exists to this day.

U.S. Pat. No. 5,543,111 describes the treatment of infectious medical materials with a radio-frequency electric field. Said treatment takes place for large quantities of wastes, in a centralized manner and in a big plant. It thus fail to meet the invention purpose, namely to treat wastes on site, as set forth below, with a portable apparatus.

Other method or apparatus of treatment are known, as for instance described in FR 2,115,951 for drying and sterilizing poultry dejection, or as described in WO 92/04920 for processing medical waste. These are all concerned with continuous processes or apparatuses, using conveyor belts or screw conveyors; processing is not batch by batch and stepwise. Plants are also large and cumbersome, and therefore unsuitable for on site treatment.

### SUMMARY OF THE INVENTION

An object of the invention is to resolve the problem of infectious wastes such as medical and veterinary wastes by proposing a method of on site treatment, comprising the steps consisting of, in order:

preparing the wastes in view of their ulterior handling and for subsequent treatment by microwave heating,

dividing the wastes into homogenous batches of substantially equal sizes,

sterilizing the waste batches using a heat source generated by microwaves in the range of 0.8 to 9 GHz, said microwaves being conveyed by wave-guides, and

transporting the sterilized wastes out of the treatment zone and preparing them to be evacuated.

A further object of the invention is a method for the on site treatment of infectious wastes, in which the microwaves are conveyed by wave-guides so as to cause said microwaves to interfere over the waste batches.

Another object of the invention is a method where small or medium size batches of wastes are treated one by one, thus allowing the method to be used on site and to be implemented on a portable or rolling apparatus.

Another object of the invention is a method in which wastes in batches are sterilized in a confined manner.

Preferably, the range of frequency for heating microwaves is comprised between 0.8 and 3 GHz, for instance 0.9 GHz or 2.455 GHz, which are currently accepted microwave frequencies for domestic and industrial uses in the United States of America and Europe, respectively.

Preparation and division of wastes may be made together, in a single step, and transportation of the sterilized wastes after treatment may be made aseptically, without exposure to the outside.

The method finds a special interest in the domain of the elimination of infectious wastes, but it also applies to other wastes, so that, unless they represent the near-totality of wastes compared to infectious wastes, it will generally not be necessary to separate in advance the infectious wastes from the non-infectious wastes. They will thus be treated without distinction, avoiding a costly upstream sorting operation.

Preferably, the preparation of the wastes is conducted in a granulating grinder, a screw compactor or a blade shredder.

If, in contrast to human or animal wastes or blood, the wastes contain low humidity, for example if they consist essentially of surgical gloves, syringes, compresses, disposable dirty laundry, etc., the preparation of the wastes will advantageously comprise a step of humidification or wetting with water or an aqueous solution in order to confer a global water content allowing an efficient and fast action of electromagnetic waves. The added aqueous solution may contain a disinfectant or an antiseptic.

The heating is carried out by the application of microwaves in the range of 0.8 GHz to 9 GHz, by contrast to what is taught in U.S. Pat. No. 5,543,111 wherein the radio frequencies used are said to be in the range of 500 KHz to 600 MHz, preferably 10 to 100 MHz.

These crucial differences, both in terms of sizes and frequencies, are critical. In U.S. Pat. No. 5,543,111, typical sizes of the apparatuses are 18 meters long, 4 meters wide and 3 meters tall and ½ wavelength of the field is comparable to one of the larger dimensions. Thus, although higher frequency resonances are said to be possible, only an oscillating electric field can be obtained over the waste batches, without propagation of electromagnetic waves.

According to the invention, where wastes are treated in individual small size batches, the radiating frequencies in combination with the additional use of wave-guides causes the microwaves to interfere and to establish an operating mode of stationary waves with maxima (peaks) and minima well defined, located and standing. Heating is not uniform, but at its maximum on a peak of interfering waves, batches of wastes are of course positioned in such maxima.

As indicated above, the handling of wastes within the apparatus is done by individual discrete charges, i.e. by batches, one by one. Each batch is disposed and confined in an individual container or cup. Thus temperatures reached upon heating within the individual containers, which are hermetically sealed during the heating step, are above 160 to 170° C. and above 4 bars, generally comprised between 160 and 200° C. with pressures ranging typically from 4 to 10 bars.

The sterilized wastes are conveyed out of the heating zone in view of their transport, for example by ejection, which is advantageously carried out by pneumatic, hydraulic or electrical means.

For the heating to sterilize the wastes, a process such as the one described in the specification of EP 0,136,453 can be used preferably. In such a process the microwave heating is generated by means of at least dual irradiation, in which for a directed distribution of heating, the irradiations form a sum field and are superimposed in the object to be heated and that the object is positioned in the region of a maximum standing wave of the sum field.

Microwaves are generated by one magnetron and split into at least two parts by wave-guides, in order to cause them to positively interfere, namely to form a sum field, over the batch of waste to be heated.

For batches of wastes having large dimensions in height, two or more magnetrons, preferably identical, with two corresponding wave-guides can be associated, the electromagnetic fields produced by each magnetron being superimposed to the other.

For further information concerning the electromagnetic wave application means, we shall refer advantageously to said specification EP 0,136,453, which is incorporated herein by reference.

According to the invention, microwaves can also be generated by at least two magnetrons generating coherent waves, which are then caused to positively interfere by the wave-guides over the batch of waste to be heated.

The invention is also relating to an apparatus for the implementation of the above method, comprising:

a preparation station for the wastes in view of their further handling and for their subsequent treatment by electromagnetic heating,

a station where the wastes are divided into homogenous batches of substantially equal sizes,

a station where the batches of waste are sterilized by a heat source by applying microwaves through wave-guides in the range of 0.8 to 9 GHz, preferably between 0.8 and 3 GHz, and

a station for the evacuation of sterilized wastes outside the treatment zone, if desired aseptically without exposure to the outside.

Advantageously, the apparatus according to the invention comprises a rotary disc wherein each stopping position corresponds to as many stations, as indicated above.

For the microwave heating to sterilize the wastes, one of the apparatuses described in the specification EP 0,136,453 is preferably used.

Divided wastes may be poured into containers and the hermetic closure required to reach the desired pressure (and temperatures) during heating is then obtained within containers, which are hermetically capped, and are in turn disposed in corresponding cavities. Divided wastes may also be treated without use of containers by being poured directly in said cavities. In this case, the hermetic closure in the cavities is reached by a sealing ring disposed in the heating station.

For batches of 0.3 to 0.5 liters, the higher range of frequency is preferably used, whereas for batches of 2 to 3 liters, the lower range is better. The smaller the batch size is, the higher the frequencies used and vice versa.

### DESCRIPTION OF THE DRAWINGS

The invention will be better understood by reference to the attached drawings, given by way of non-limiting example, wherein, in cross-section:

FIG. 1 is an elevation view of the apparatus according to the invention;

FIG. 2 is a side view of same apparatus; and

FIG. 3 is a top view of same apparatus.

### DESCRIPTION OF THE PREFERRED EMBODIMENTS

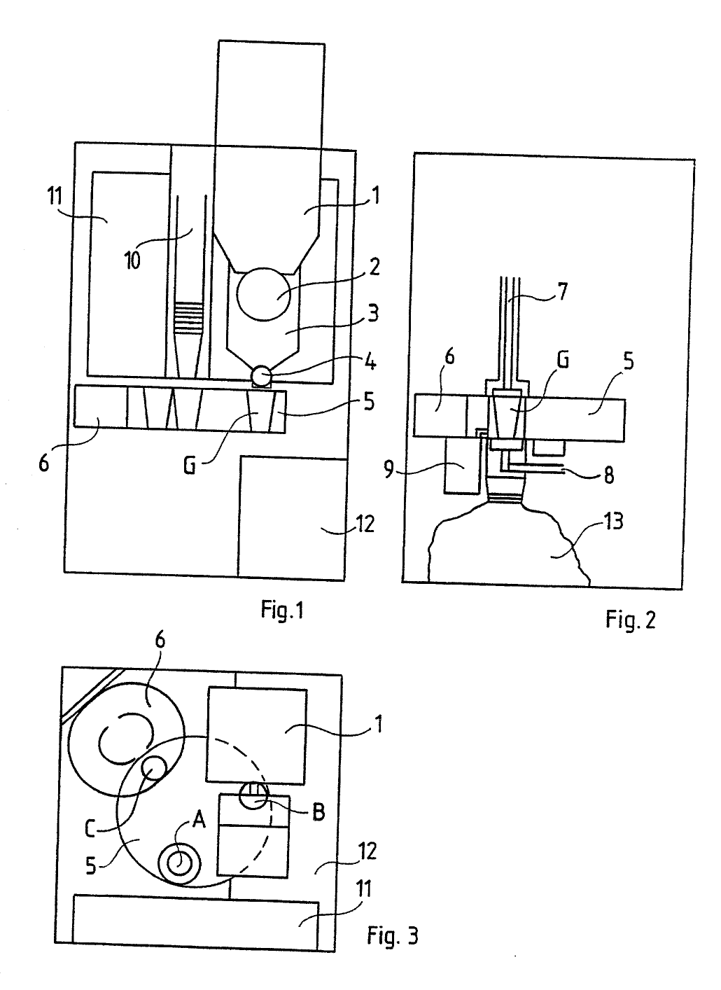

As seen on FIG. 1, which is a side view, an apparatus according to the invention comprises a feed hopper 1 under which is situated a motor-driven granulator 2 which leads to a buffer lock 3, which feeds in its turn a motor driven metering device 4 filling the containers G from above.

In a non illustrated alternative, the feed hopper is provided with a waste compactor allowing a more regular and more homogenous operation of the granulator 2. This compactor can take for example the form of two flaps articulated on their bases against the side walls of the hopper 1, laying at rest against these side walls, and swingable towards one another inwards to substantially join end-to-end, thus pushing and pressing the wastes towards the granulator 2.

These containers G, received in cavities, will thus be filled with wastes in view of their treatment by electromagnetic waves. As it can be seen on the Figure, the cup-shaped containers G, are supplied from a container distributor 10 and, once filled with wastes by means of the metering device 4, the cavities closed hermetically, and these containers are directed towards wave-guide 6 of the electromagnetic wave application means. Alternatively, the containers can be in the form of a film, for example a polyethylene film, disposed in each cavity, which film is filled with wastes, and is closed by welding once full. As in the previously described embodiment, these containers are then directed towards the wave-guide 6 of the electromagnetic wave application means, after each cavity has been hermetically closed. This wave-guide 6 is formed so as to allow interference of microwaves emitted by the magnetron.

As shown in this Figure, there is a tank 11 filled with water or an aqueous solution to which a small quantity of disinfectant can be added, the aqueous solution in question dampening or wetting the granulated wastes at the level of the metering device 4. Reference 12 represents schematically the electrical supply of the unit, as well as a compressor.

FIG. 2, where the same reference numbers represent the same elements as in FIG. 1, shows a piston 7 which, once the sterilization by electromagnetic waves is finished, will eject the container G in a hermetic bag 13 in which the sterilized wastes will be transported towards the exterior. FIG. 2 also depicts a container support 8 for introduction in the apparatus, which can be shifted aside for container ejection and the magnetron 9.

As can be seen on the FIG. 3 where the same reference numbers represent the same elements as in FIGS. 1 and 2, the machine is constituted in such a way that the operations are undertaken at stations organized around a rotary platen 5 containing cavities machined in its body for receiving the containers G. These stations are disposed at 120° and each rotation by this angle allows a container G to be presented either to the filling (station B), or under a magnetron of the electromagnetic wave sterilizing device (station C), or lastly in front of the ejection piston (station A). Station C is entirely housed inside the wave-guide 6.

In another embodiment of the apparatus according to the invention, not illustrated, the rotating stations are replaced by a drawer slide system with a reciprocal movement in and out of the heating zone. No containers are used to restrain the divided wastes. Said divided wastes are simply disposed directly into a cavity having for instance a form of a cylindrical or trunc-conical shape and conveyed to the heating station. Before heating said cavity is hermetically sealed against the main body of the apparatus by means of a sealing ring.

Although the invention has been described with particular reference to dispose of medical or veterinary wastes, it is usable for any type of wastes, where it represents a major improvement by delivering finely divided and regular harmless end products, in an efficient and inexpensive manner.

### EXAMPLE 1

The apparatus described above has been used to sterilize bulk wastes originating from the operating room of a care unit, by means of an electromagnetic wave apparatus as described in the above-mentioned EP 0,136,453, the wastes having been previously reduced to homogenous particles with dimensions in the range of 1-5 mm. At the time of feeding into the containers, an aqueous solution was sprayed on the waste in order to obtain a weight proportion of water relative to the waste of 1:2.

The application of electromagnetic waves having a frequency of 2.455 Ghz provokes, inside the hermetically closed cavities wherein the containers G are contained, a rapid increase of temperature and of pressure, reaching an upper temperature of 165° C. at a pressure of 6 bars. Containers G are cylindrical with a volume of 350 ml and a height of 110 mm. Heating stationary time of a batch is typically, for raw hospital waste, around 2 minutes for an electromagnetic field applied by one magnetron of 1.2 kW.

These pressure and temperature conditions guarantee that the waste is effectively sterilized.

### EXAMPLE 2

The same treatment is repeated by using the same frequency of 2.455 Mhz to obtain a sterilized end product. In this case, the heating stationary time of a batch is typically, for same raw hospital waste, around 1 minute for an electromagnetic field, the frequency being applied by two superimposed magnetrons of 1.2 kW each.

The process according to the invention presents the enormous advantage of an on site or in situ treatment, without exterior transportation of potentially infectious waste and thus without risking accidental contamination of the environment. The virulence and the dangers of some bacterial or viral strains infecting the diseased or infesting hospitals being well known, it is easy to appreciate the progress brought by the invention.

Furthermore, the apparatus according to the invention may have restricted dimensions, for example allowing it to pass through doors. Each care unit in a clinic or a hospital could thus have its own apparatus, which could be placed at selected focal points, for example near operating rooms, allowing a great flexibility in the management of wastes. A centralized management of wastes can thus be avoided, with all the risks it involves due to the volumes to be treated and the ever-present possibility of material failure and human errors.

I claim:

1. A method for the on site treatment of infectious wastes such as medical and veterinary wastes, comprising the steps of:
preparing the wastes in view of their ulterior handling and for subsequent treatment by microwave heating,
dividing the wastes in homogenous batches of substantially equal sizes,
sterilizing the waste batches using a heat source generated by microwaves in the range of 0.8 to 9 GHz, said microwaves being conveyed by wave guides,
transporting the sterilized wastes out of the treatment zone, and
collecting the wastes to be evacuated.

2. A method according to claim 1, further comprising conveying the microwaves by wave-guides so as to cause said microwaves to interfere over the waste batches.

3. A method according to claim 1, wherein the step of preparing the wastes further comprises grinding in an apparatus selected from the group of a granulating grinder, a screw compactor and a blade shredder.

4. A method according to claim 1, wherein the steps of preparing the wastes and dividing the wastes are combined into a single step.

5. A method according to claim 1, wherein the step of transporting the sterilized wastes is made aseptically, without exposure to the outside.

6. A method according to claim 1, wherein the step of preparing the wastes further comprises humidifying or wetting the wastes with at least one of water and an aqueous solution.

7. A method according to claim 1, wherein the treatment takes place in a confined manner at a temperature greater than 160° C. and under a pressure greater than 4 bars.

8. A method according to claim 1, wherein each batch is disposed in an individual container.

9. A method according to claim 1, wherein each batch is poured into a cavity.

10. A method according to claim 1, wherein said microwaves are generated by one magnetron and split into at least parts by wave-guides, in order to cause them to positively interfere over the batch of waste to be heated.

11. A method according to claim 10, wherein said microwaves are generated by at least two associated magnetrons with at least two corresponding wave-guides, said magnetrons being superimposed.

12. A method according to claim 1, wherein said microwaves are generated by at least two magnetrons generating coherent waves which are then caused to positively interfere by the wave-guides over the batch of waste to be heated.

13. An apparatus for the implementation of the method for the on site treatment of infectious wastes such as medical and veterinary wastes, comprising:
a preparation station for the wastes in view of their further handling and for their subsequent treatment by microwave heating;
a division station where the wastes are divided into homogenous batches of substantially equal sizes;
a sterilization station where the batches of waste are sterilized by a heat source by means generating microwaves in the range of 0.8 to 9 GHz, said microwaves being conveyed by wave guides; and
a removal station for the evacuation of sterilized waste outside the treatment zone.

14. An apparatus according to claim 13, wherein the preparation station and the division station are a single station.

15. An apparatus according to claim 13, further comprising a rotatable platen having at least one cavity for receiving a container presenting, at each rotation, said container to one of said stations.

16. An apparatus according to claim 13, further comprising a sliding drawer containing a cavity and having reciprocal movement into and out of said sterilization station.
